(19) United States
(12) Patent Application Publication
(10) Pub. No.: US 2021/0322221 A1
(43) Pub. Date: Oct. 21, 2021

(54) VACUUM-ASSISTED PROTECTIVE EYEWEAR

(71) Applicants: Stephen Damm, Zionsville, IN (US); Tyler Schultz, Indianapolis, IN (US); Daniel Schmidt, Carmel, IN (US); Daniel Biehl, Pendleton, IN (US); Matthew Green, Greenwood, IN (US)

(72) Inventors: Stephen Damm, Zionsville, IN (US); Tyler Schultz, Indianapolis, IN (US); Daniel Schmidt, Carmel, IN (US); Daniel Biehl, Pendleton, IN (US); Matthew Green, Greenwood, IN (US)

(21) Appl. No.: 16/852,434

(22) Filed: Apr. 18, 2020

(51) Int. Cl. A61F 9/02

(52) U.S. Cl. CPC A61F 9/025; A61F 9/028; A61F 9/029

(57) ABSTRACT

An eye protection apparatus is disclosed which includes a frame, one or more nozzles, one or more lenses, one or more attachment members, and one or more channels adapted to receive a negative pressure.

### TECHNICAL FIELD

The present disclosure generally relates to eye protection, and in particular, to a vacuum-assisted eye protection device.

### BACKGROUND

This section introduces aspects that may help facilitate a better understanding of the disclosure. Accordingly, these statements are to be read in this light and are not to be understood as admissions about what is or is not prior art.

The following disclosure deals with protective eyewear. Many different jobs require the use of eye protection and many standards are in place based on the job.

When working with power tools or dangerous materials, the safety of the user is of the utmost importance. Various devices have been created to protect the worker's most sensitive areas such as the eyes, mouth, and ears.

Regular safety glasses are limited by their design. Particles can easily fly around the edges into the operator's eye. Goggles are another form of eye protection that seal the eyes better but are not feasible in all situations, as the sides could block peripheral vision.

Eye protection with positive pressure generating devices that propel particles away from the eye exists as well. This idea helps to keep particles away from the eye but does not control where the particles go. The particles could be blown directly towards one's mouth or towards whatever the user may be working on. Breathing in hazardous particles can be severely harmful as well.

Therefore, there is an unmet need for a novel approach to protective eyewear.

### SUMMARY

An eye protection apparatus is disclosed which includes a frame, one or more nozzles, one or more lenses, one or more attachment members, and one or more channels adapted to receive a negative pressure.

An eye protection retrofit kit for an existing eyewear is also disclosed which includes one or more nozzles, a coupler, and one or more channels adapted to receive a negative pressure.

An eye protection system is also disclosed which include a frame, one or more nozzles; one or more lenses, one or more attachment members, one or more channels, and a negative pressure source.

### BRIEF DESCRIPTION OF DRAWINGS

FIG. 1 is a perspective view of an eye protection apparatus according to the present disclosure;

FIG. 2 is a front view of the eye protection apparatus of FIG. 1;

FIG. 3 is a side view of the eye protection apparatus of FIG. 1;

FIG. 4 is a perspective view of an eye protection attachment apparatus for eyewear according to the present disclosure;

FIG. 5 is a perspective view of the eye protection system according to the present disclosure; and

FIG. 6 is a side view of the system of FIG. 5;

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure, the term “about” can allow for a degree of variability in a value or range, for example, within 10%, within 5%, or within 1% of a stated value or of a stated limit of a range.

In the present disclosure, the term “substantially” can allow for a degree of variability in a value or range, for example, within 90%, within 95%, or within 99% of a stated value or of a stated limit of a range.

A novel idea to an eye protection device will capture the particles around the face and dispense of them through a channel using negative pressure. Additionally, the device may be attached to a pre-existing eyewear.

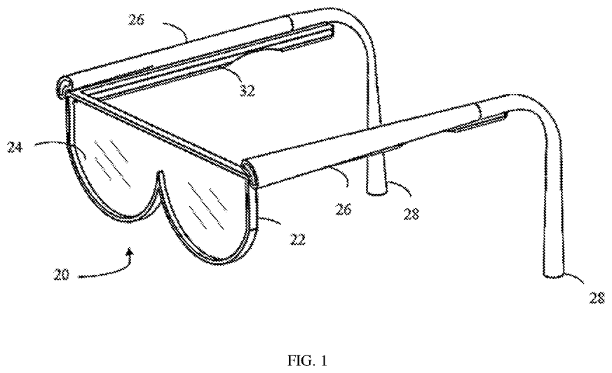

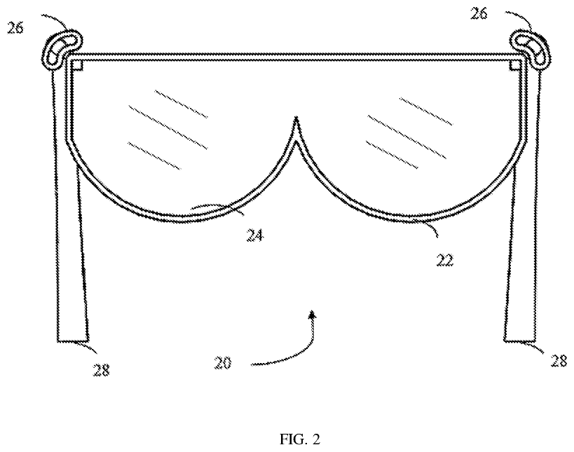

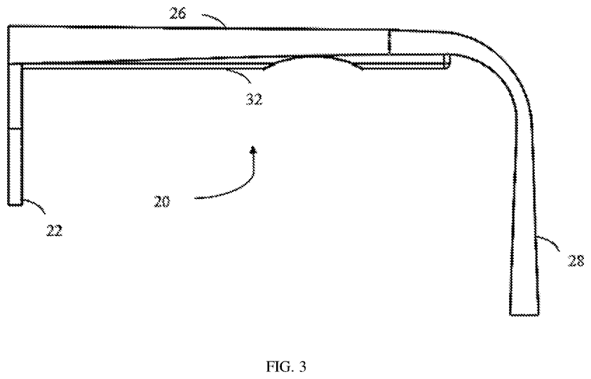

Referring to FIG. 1, FIG. 2, and FIG. 3, a protective eyewear apparatus 20 is shown according to the present disclosure, in which a frame 22 is configured to fit on a user's face over the user's eyes. A lens 24 which is connected to the frame 22, where the lens may be connected by being snapped into a grooved frame or where the frame may be screwed down over the lens to secure the lens in place. There can be one or more lenses 24 that can be used to fit the shape of the frame better. A nozzle 26 is connected to the frame 22 or the attachment member 32 where the nozzle entrance is pointed towards the area where the user's face is located and the nozzle exit may be located towards the side, top, or rear of the user's head. There may be more than one nozzle 26 to fit the contour of the frame better as well as to increase the area of suction around the frame where more particles around the face area may be captured. The multiple nozzles can be coupled to each other where the nozzle exit can be coupled to another nozzle exit in series to have one nozzle exit for multiple nozzles. The nozzle may be connected by a coupler, fastener, adhesive or even built into the frame 22 or attachment member 32 as one piece. One or more attachment members 32 is attached to the frame 22 by an adhesive, a hinge, built into, or molded with the frame. The attachment members can be, but not limited to, an elastic band or a rigid member (also referred to as the temples of a pair of glasses). A channel 28 is connected to the nozzle 26, specifically to the nozzle exit. If there are multiple nozzles 26, there may be multiple channels 28 connected to the nozzle exits. The channels 28 may be coupled together where multiple channels are linked to one channel opening. The channel may be a tube wherein the tube is connected to the nozzle with the tube being slightly bigger than the nozzle exit where the tube can be slid over and fitted onto the nozzle. The tube may also be clamped over the nozzle with a fitting. The tube may also be smaller than the nozzle exit where the tube may be fitted inside of the nozzle. The channel may also be inside of the attachment member 32. The attachment member 32 may be hollow wherein the channel is the hollowed out portion.

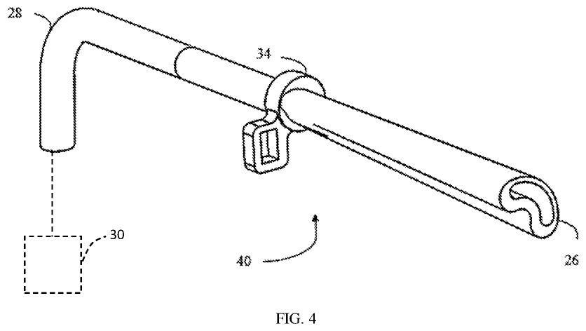

Referring to FIG. 4, an eye protection attachment apparatus 40 which is intended to be used as a retrofit kit for existing eyewear is shown according to the present disclosure. The eye protection attachment apparatus 40 where a channel 28 is connected to the nozzle 26. Attached to the nozzle 26 is a coupler 34 that can couple the eye protection attachment apparatus 40 to an already existing piece of eyewear's frame or attachment members 32. The existing piece of eyewear can be, but not limited to, a pair of glasses or goggles.

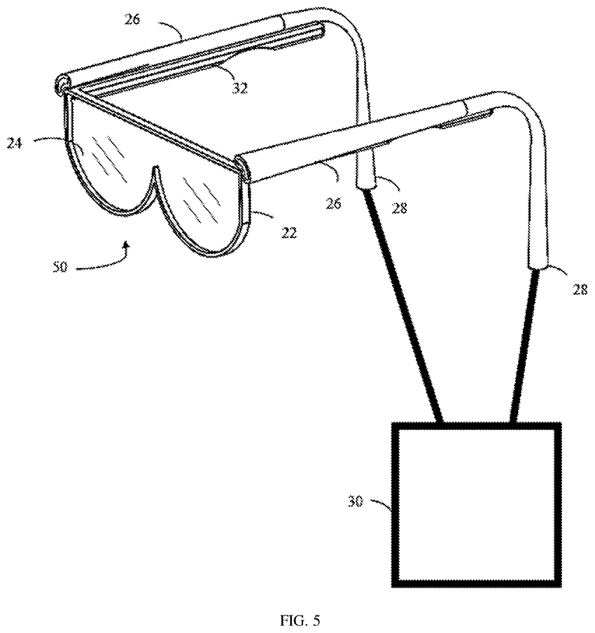

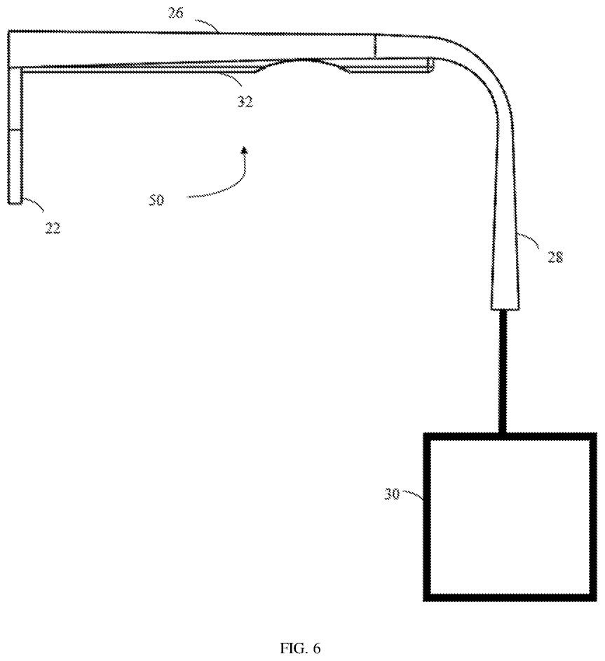

Referring to FIG. 5 and FIG. 6, a protective eyewear system 50 is shown according to the present disclosure. The protective eyewear system 50 includes the same elements as shown in the protective eyewear apparatus 20 while including the negative pressure source 30. The channel 28 is connected to a negative pressure source 30. The negative pressure source may be, but not limited to, a vacuum operating between a pressure of about 8 PSIA to about 11 PSIA. There may be more than one channel 28 that is connected to the negative pressure source 30.

Those having ordinary skill in the art will recognize that numerous modifications can be made to the specific implementations described above. The implementations should not be limited to the particular limitations described. Other implementations may be possible.

What is claimed is:

1. An eye protection apparatus comprising:
a frame;
one or more nozzles;
one or more lenses;
one or more attachment members; and
one or more channels adapted to receive a negative pressure.

2. The eye protection apparatus from claim 1, wherein the one or more lenses are coupled to the frame.

3. The eye protection apparatus of claim 1, wherein the one or more channels are coupled to the one or more nozzles.

4. The eye protection apparatus of claim 1, wherein the one or more nozzles are coupled to the frame.

5. The eye protection apparatus of claim 1, wherein the one or more attachment members are coupled to the frame.

6. The eye protection apparatus of claim 1, further comprising a negative pressure source coupled to the one or more channels.

7. The eye protection apparatus of claim 6, wherein the pressure of the negative pressure source is between about 8 PSIA and about 11 PSIA.

8. An eye protection retrofit kit for an existing eyewear, comprising:
one or more nozzles;
a coupler; and
one or more channels adapted to receive a negative pressure.

9. The eye protection retrofit kit for an existing eyewear of claim 8, wherein the nozzle is fluidly coupled to the channel.

10. The eye protection retrofit kit for an existing eyewear of claim 8, wherein the coupler is coupled to the nozzle and adapted to attach to an eyewear apparatus.

11. The eye protection retrofit kit for an existing eyewear of claim 8, further comprising a negative pressure source coupled to the one or more channels.

12. The eye protection retrofit kit for an existing eyewear of claim 11, wherein the pressure of the negative pressure source is between about 8 PSIA and about 11 PSIA.

13. An eye protection system comprising:
a frame;
one or more nozzles;
one or more lenses;
one or more attachment members;
one or more channels, and
a negative pressure source.

14. The eye protection system of claim 13, wherein the negative pressure source is coupled to the one or more channels.

15. The eye protection system of claim 13, wherein the one or more lenses are coupled to the frame.

16. The eye protection system of claim 13, wherein the one or more channels are coupled to the one or more nozzles.

17. The eye protection system of claim 13, wherein the one or more nozzles are coupled to the frame.

18. The eye protection system of claim 13, wherein the pressure of the negative pressure source is between about 8 PSIA and about 11 PSIA.
